(19) United States
(12) Patent Application Publication
(10) Pub. No.: US 2009/0005391 A1
(43) Pub. Date: Jan. 1, 2009

(54) Tricyclo Substituted Amides

(76) Inventors: Matthew Colin Thor Fyfe, Oxfordshire (GB); Martin James Procter, Oxfordshire (GB)

Correspondence Address: ELI LILLY & COMPANY, PATENT DIVISION, P.O. BOX 6288, INDIANAPOLIS, IN, 46206-6288, US

(21) Appl. No.: 12/091,947

(22) PCT Filed: Nov. 3, 2006
(86) PCT No.: PCT/EP2006/068087

§ 371 Date: Apr. 29, 2008

(30) Foreign Application Priority Data
Nov. 3, 2005 (GB) 0522455.5
Feb. 16, 2006 (GB) 0603129.8

(51) Int. Cl. A61K 31/4433; A61P 3/00; A61P 3/10; C07D 405/12; C07D 413/12; C07D 417/12; A61K 31/497; A61K 31/506; A61K 31/4155; A61K 31/501; A61K 31/427; A61K 31/422

(52) U.S. Cl. 514/252.01; 514/256; 544/336; 546/282.1; 548/246; 548/195; 544/238; 548/365.7; 544/329; 514/407; 514/371; 514/380; 514/336; 514/255.05

(57) ABSTRACT

Compounds of Formula (I) or pharmaceutically acceptable salts thereof, are useful in the prophylactic and therapeutic treatment of hyperglycemia and diabetes.

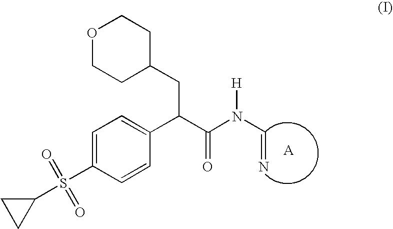

### BACKGROUND OF THE INVENTION

The present invention is directed to tri(cyclo) substituted amide compounds. In particular, the present invention is directed to amide compounds substituted i) at the carbonyl carbon with an ethyl attached to a phenyl ring and a heterocyclic ring, and ii) at the amino with a nitrogen bearing heteroaryl ring, which are modulators of glucokinase and are useful in the prophylactic or therapeutic treatment of hyperglycemia and diabetes, particularly type II diabetes.

Glucokinase (“GK”) is believed to be important in the body's regulation of its plasma glucose level. GK, found principally in the liver and pancreas, is one of four hexokinases that catalyze the initial metabolism of glucose. The GK pathway is saturated at higher glucose levels than the other hexokinase pathways (see R. L. Printz et al., Annu. Rev. Nutr., 13:463-496 (1993)). GK is critical to maintaining the glucose balance in mammals. Animals that do not express GK die soon after birth with diabetes, while animals that overexpress GK have improved glucose tolerance. Activation of GK can lead to hyperinsulinemic hypoglycemia (see, for example, H. B. T. Christesen et al., Diabetes, 51:1240-1246 (2002)). Additionally, type II maturity-onset diabetes of the young is caused by the loss of function mutations in the GK gene, suggesting that GK operates as a glucose sensor in humans (Y. Liang et al., Biochem. J., 309:167-173 (1995)). Thus, compounds that activate GK increase the sensitivity of the GK sensory system and would be useful in the treatment of hyperglycemia, particularly the hyperglycemia associated with type II diabetes. It is therefore desirable to provide novel compounds that activate GK to treat diabetes, in particular compounds which demonstrate improved properties desirable for pharmaceutical products compared to known GK activators.

International Patent Publication No. WO2001/044216 and U.S. Pat. No. 6,353,111 describe (E)-2,3-disubstituted-N-heteroarylacrylamides as GK activators. International Patent Publication No. WO2002/014312 and U.S. Pat. Nos. 6,369,232, 6,388,088, and 6,441,180 describe tetrazolylphenylacetamide GK activators. International Patent Publication No. WO2000/058293, European Patent Application No. EP 1169312 and U.S. Pat. No. 6,320,050 describe arylcycloalkylpropionamide GK activators. International Patent Publication No. WO2002/008209 and U.S. Pat. No. 6,486,184 describe alpha-acyl and alpha-heteroatom-substituted benzene acetamide GK activators as anti-diabetic agents. International Patent Publication No. WO2001/083478 describes hydantoin-containing GK activators. International Patent Publication No. WO2001/083465 and U.S. Pat. No. 6,388,071 describe alkynylphenyl heteroaromatic GK activators. International Patent Publication No. WO2001/085707 and U.S. Pat. No. 6,489,485 describe para-amine substituted phenylamide GK activators. International Patent Publication No. WO2002/046173 and U.S. Pat. Nos. 6,433,188, 6,441,184, and 6,448,399 describe fused heteroaromatic GK activators. International Patent Publication No. WO2002/048106 and U.S. Pat. No. 6,482,951 describe isoindolin-1-one GK activators. International Patent Publication No. WO2001/085706 describes substituted phenylacetamide GK activators for treating type II diabetes. U.S. Pat. No. 6,384,220 describes para-aryl or heteroaryl substituted phenyl GK activators. French Patent No. 2,834,295 describes methods for the purification and crystal structure of human GK. International Patent Publication No. WO2003/095438 describes N-heteroaryl phenylacetamides and related compounds as GK activators for the treatment of type II diabetes. U.S. Pat. No. 6,610,846 describes the preparation of cycloalkylheteroaryl propionamides as GK activators. International Patent Publication No. WO2003/000262 describes vinyl phenyl GK activators. International Patent Publication No. WO2003/000267 describes aminonicotinate derivatives as GK modulators. International Patent Publication No. WO2003/015774 describes compounds as GK modulators. International Patent Publication No. WO2003047626 describes the use of a GK activator in combination with a glucagon antagonist for treating type II diabetes. International Patent Publication No. WO2003/055482 describes amide derivatives as GK activators. International Patent Publication No. WO2003/080585 describes aminobenzamide derivatives with GK activity for the treatment of diabetes and obesity. International Patent Publication No. WO2003/097824 describes human liver GK crystals and their used for structure-based drug design. International Patent Publication No. WO2004/002481 discloses arylcarbonyl derivatives as GK activators. International Patent Publication Nos. WO2004/072031 and WO2004/072066 disclose tri(cyclo) substituted amide compounds as GK activators. International Patent Application PCT/GB2005/050129 (published after the priority date of the present application) discloses amide compounds substituted i) at the carbonyl carbon with an ethyl/ethenyl attached to a phenyl ring and a carbocyclic ring, and ii) at the amino with a nitrogen bearing heteroaryl or unsaturated heterocyclyl ring, which are modulators of glucokinase and are useful in the prophylactic or therapeutic treatment of hyperglycemia and diabetes, particularly type II diabetes.

The present invention provides novel GK activators which may demonstrate improved properties desirable for pharmaceutical products compared to known GK activators, such as increased potency, increased in vivo efficacy and/or longer half-life.

### SUMMARY OF THE INVENTION

Compounds represented by Formula (I):

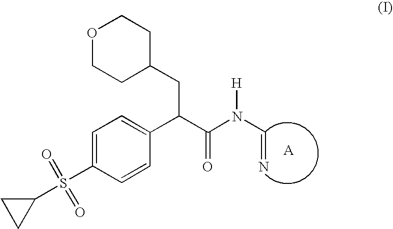

or pharmaceutically acceptable salts thereof, are useful in the prophylactic or therapeutic treatment of hyperglycemia and diabetes, particularly type II diabetes.

### DETAILED DESCRIPTION OF THE INVENTION

The present invention is directed to compounds of Formula (I):

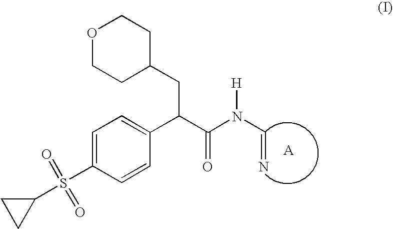

wherein A is a nitrogen containing heteroaryl ring selected from 5-methylpyrazin-2-yl, 5-methylpyrid-2-yl, 5-chloropyrid-2-yl, pyrid-2-yl, 5-methylisoxazol-3-yl, isoxazol-3-yl, 5-methylthiazol-2-yl, 6-methylpyridazin-3-yl, 1-methylpyrazol-3-yl and pyrimidin-4-yl;

and pharmaceutically acceptable salts thereof.

A is preferably 5-methylpyrazin-2-yl, 5-methylpyrid-2-yl, 5-chloropyrid-2-yl, pyrid-2-yl or 5-methylthiazol-2-yl, more preferably 5-methylpyrazin-2-yl or pyrid-2-yl, especially 5-methylpyrazin-2-yl.

In one embodiment of the present invention A represents 5-methylpyrazin-2-yl:

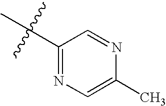

In a second embodiment of the present invention A represents 5-methylpyrid-2-yl:

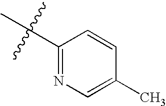

In a third embodiment of the present invention A represents 5-chloropyrid-2-yl:

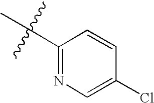

In a fourth embodiment of the present invention A represents pyrid-2-yl:

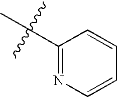

In a fifth embodiment of the present invention A represents 5-methylisoxazol-3-yl:

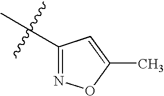

In a sixth embodiment of the present invention A represents isoxazol-3-yl:

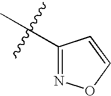

In a seventh embodiment of the present invention A represents 5-methylthiazol-2-yl:

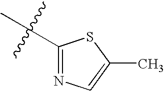

In an eighth embodiment of the present invention A represents 6-methylpyridazin-3-yl:

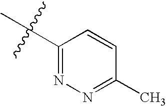

In a ninth embodiment of the present invention A represents 1-methyl-pyrazol-3-yl:

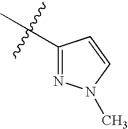

In a tenth embodiment of the present invention, A represents 4-pyrimidinyl:

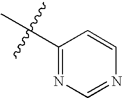

The carbon atom linking the phenyl ring and the tetrahydropyran containing sidechain to the amide carbonyl carbon is a chiral centre. Accordingly, at this centre, the compound may be present either as a racemate or as a single enantiomer in the (R)- or (S)-configuration. The (R)-enantiomers are preferred.

The term “pharmaceutically acceptable salts” includes salts prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, methanesulfonic, and tartaric acids.

When the compound of the above formulae and pharmaceutically acceptable salts thereof exist in the form of solvates or polymorphic forms, the present invention includes any possible solvates and polymorphic forms. The type of solvent that forms the solvate is not particularly limited so long as the solvent is pharmacologically acceptable. For example, water, ethanol, propanol, acetone or the like can be used.

Since the compounds of Formula (I) are intended for pharmaceutical use they are preferably provided in substantially pure form, for example at least 60% pure, more suitably at least 75% pure, at least 95% pure and especially at least 98% pure (% are on a weight for weight basis).

The invention also encompasses a pharmaceutical composition that is comprised of a compound of Formula (I), or a pharmaceutically acceptable salt thereof, in combination with a pharmaceutically acceptable carrier.

Preferably the composition is comprised of a pharmaceutically acceptable carrier and a non-toxic therapeutically effective amount of a compound of Formula (I), or a pharmaceutically acceptable salt thereof.

Moreover, within this embodiment, the invention encompasses a pharmaceutical composition for the prophylaxis or treatment of hyperglycemia and diabetes, particularly type II diabetes, by the activation of GK, comprising a pharmaceutically acceptable carrier and a non-toxic therapeutically effective amount of compound of Formula (I), or a pharmaceutically acceptable salt thereof.

The invention also provides the use of a compound of Formula (I), or a pharmaceutically acceptable salt thereof, as a pharmaceutical.

The compounds and compositions of the present invention are effective for treating hyperglycemia and diabetes, particularly type II diabetes, in mammals such as, for example, humans.

The invention also provides a method of prophylactic or therapeutic treatment of a condition where activation of GK is desirable comprising a step of administering an effective amount of a compound of Formula (I), or a pharmaceutically acceptable salt thereof.

The invention also provides a method of prophylactic or therapeutic treatment of hyperglycemia or diabetes, particularly type II diabetes, comprising a step of administering an effective amount of a compound of Formula (I), or a pharmaceutically acceptable salt thereof.

The invention also provides a method for the prevention of diabetes, particularly type II diabetes, in a human demonstrating pre-diabetic hyperglycemia or impaired glucose tolerance comprising a step of administering an effective prophylactic amount of a compound of Formula (I), or a pharmaceutically acceptable salt thereof.

The invention also provides the use of a compound of Formula (I), or a pharmaceutically acceptable salt thereof, as a GK activator.

The invention also provides the use of a compound of Formula (I), or a pharmaceutically acceptable salt thereof, for the prophylactic or therapeutic treatment of hyperglycemia or diabetes, particularly type II diabetes.

The invention also provides the use of a compound of Formula (I), or a pharmaceutically acceptable salt thereof, for the prevention of diabetes, particularly type II diabetes, in a human demonstrating pre-diabetic hyperglycemia or impaired glucose tolerance.

The invention also provides the use of a compound of Formula (I), or a pharmaceutically acceptable salt thereof, in the manufacture of a medicament for the activation of GK.

The invention also provides the use of a compound of Formula (I), or a pharmaceutically acceptable salt thereof, in the manufacture of a medicament for the prophylactic or therapeutic treatment of hyperglycemia or diabetes, particularly type II diabetes.

The invention also provides the use of a compound of Formula (I), or a pharmaceutically acceptable salt thereof, in the manufacture of a medicament for the prevention of diabetes, particularly type II diabetes, in a human demonstrating pre-diabetic hyperglycemia or impaired glucose tolerance.

The compounds and compositions of the present invention may be optionally employed in combination with one or more other anti-diabetic agents or anti-hyperglycemic agents, which include, for example, sulfonylureas (e.g. glyburide, glimepiride, glipyride, glipizide, chlorpropamide, gliclazide, glisoxepid, acetohexamide, glibornuride, tolbutamide, tolazamide, carbutamide, gliquidone, glyhexamide, phenbutamide, tolcyclamide, etc.), biguanides (e.g. metformin, phenformin, buformin, etc.), glucagon antagonists (e.g. a peptide or non-peptide glucagon antagonist), glucosidase inhibitors (e.g. acarbose, miglitol, etc.), insulin secetagogues, insulin sensitizers (e.g. troglitazone, rosiglitazone, pioglitazone, etc.) and the like; or anti-obesity agents (e.g. sibutramine, orlistat, etc.) and the like. The compounds and compositions of the present invention and the other anti-diabetic agents or anti-hyperglycemic agents may be administered simultaneously, sequentially or separately.

The pharmaceutical compositions of the present invention comprise a compound of Formula (I), or a pharmaceutically acceptable salt thereof, as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, as well as administration through inhaling, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The pharmaceutical compositions according to the invention are preferably adapted for oral administration.

In practice, the compounds of Formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compounds of Formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound of Formula (I), or a pharmaceutically acceptable salt thereof. The compounds of Formula (I), or pharmaceutically acceptable salts thereof, can also be included in pharmaceutical compositions in combination with one or more other therapeutically active compounds.

The pharmaceutical compositions of this invention include pharmaceutically acceptable liposomal formulations containing a compound of Formula (I), or a pharmaceutically acceptable salt thereof.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules, and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent or other such excipient. These excipients may be, for example, inert diluents such as calcium carbonate, sodium carbonate, lactose, calcium phosphate or sodium phosphate; granulating and disintegrating agents, for example, corn starch, or alginic acid; binding agents, for example, starch, gelatin, or acacia; and lubricating agents, for example, magnesium stearate, stearic acid, or talc. The tablets may be uncoated or they may be coated by known techniques to delay disintegration and absorption in the gastrointestinal tract and thereby provide a sustained action over a longer time. For example, a time delay material such as glyceryl monostearate, or glyceryl distearate may be used.

In hard gelatin capsules, the active ingredient is mixed with an inert solid diluent, for example, calcium carbonate, calcium phosphate, or kaolin. In soft gelatin capsules, the active ingredient is mixed with water or an oil medium, for example, peanut oil, liquid paraffin, or olive oil. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95% of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage and thus, preferably should be preserved against the contaminating action of microorganisms such as bacteria and fungi. The carrier can be a solvent or dispersion medium containing, for example, water, ethanol, polyol (e.g. glycerol, propylene glycol, and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound of Formula (I), or a pharmaceutically acceptable salt thereof, via conventional processing methods. As an example, a cream or ointment is prepared by admixing hydrophilic material and water, together with about 5 wt % to about 10 wt % of the compound of Formula (I), to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

Pharmaceutical compositions of this invention can be in a form suitable for inhaled administration. Such administration can be in forms and utilizing carriers described in, for example, 1) Particulate Interactions in Dry Powder Formulations for Inhalation, Xian Zeng et al, 2000, Taylor and Francis, 2) Pharmaceutical Inhalation Aerosol Technology, Anthony Hickey, 1992, Marcel Dekker, 3) Respiratory Drug Delivery, 1990, Editor: P. R. Byron, CRC Press.

In addition to the aforementioned carrier ingredients, the pharmaceutical compositions described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of Formula (I), or a pharmaceutically acceptable salt thereof, may also be prepared in powder or liquid concentrate form.

Generally, dosage levels of the order of from about 0.01 mg/kg to about 150 mg/kg of body weight per day are useful in the treatment of the above-indicated conditions, or alternatively about 0.5 mg to about 10 g per patient per day. For example, type II diabetes may be effectively treated by the administration of from about 0.01 to 100 mg of the compound per kilogram of body weight per day, or alternatively about 0.5 mg to about 7 g per patient per day.

It is understood, however, that the specific dose level for any particular patient will depend upon a variety of factors including the age, body weight, general health, sex, diet, time of administration, route of administration, rate of excretion, drug combination and the severity of the disease in the particular diabetic patient undergoing therapy. Further, it is understood that the compounds and salts thereof of this invention can be administered at subtherapeutic levels prophylactically in anticipation of a hyperglycemic condition.

The compounds of Formula (I) may exhibit advantageous properties compared to known glucokinase activators, such properties may be illustrated in the assays described herein or in other assays known to those skilled in the art. In particular, compounds of the invention may exhibit improved values for Kₘ, Vₘₐₓ, EC₅₀, maximum activation (glucose concentration=5 mM), maximum blood glucose reduction on basal blood glucose levels and/or reduction of postprandial glucose peak in an oral glucose tolerance test (OGTT), or other advantageous pharmacological properties such as enhanced aqueous solubility, and/or enhanced metabolic stability, compared to known GK activators. The compounds of the invention may also demonstrate one or more of the following properties compared to known compounds: reduced neurotoxicity, longer duration of action (e.g. improved half-life/higher plasma protein binding), improved bioavailability, and/or increased potency (e.g. in vitro or in vivo).

### Experimental

In accordance with this invention, the compounds of Formula (I) can be prepared following the protocol illustrated in Scheme 1 below:

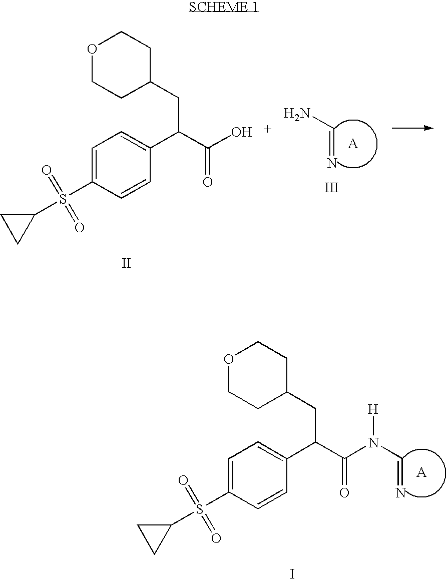

The carboxylic acid II, or an activated derivative thereof, may be condensed with the amine III, or a salt thereof, e.g. the hydrochloride salt, using a variety of coupling conditions known to those skilled in the art. For example, it is possible to condense the enantiopure carboxylic acid II with amine III, or a salt thereof, using a reagent that causes negligible racemisation, e.g. benzotriazol-1-yloxytris(pyrrolidino)phosphonium hexafluorophosphate (J. Coste et al., Tetrahedron Lett., 1990, 31, 205-208), to furnish enantiopure amides of Formula (I). Alternatively the carboxylic acid carboxylic acid II may be treated with (COCl)₂ and DMF in dichloromethane e.g. at −45° C., followed by the addition of the amine III and pyridine.

Alternatively, a racemic mixture of amides can be prepared from racemic carboxylic acid II and then separated by means of chiral high performance liquid chromatography employing a chiral stationary phase (which can be purchased from, for example, Daicel Chemical Industries, Ltd, Tokyo, Japan) to provide the desired compound of Formula (I).

The amines III are commercially available or are readily prepared using known techniques.

Preparation of the carboxylic acid II is described in WO2004/072031 (Preparation 22 therein). The racemic carboxylic acid II can be separated into R and S enantiomers by a number of means. One possible method involves the use of chiral high performance liquid chromatography employing a chiral stationary phase (which can be purchased from, for example, Daicel Chemical Industries, Ltd, Tokyo, Japan) to provide the desired compound of Formula (I). A second method involves reaction of with a chiral agent, for example a chiral oxazolidinone derivative (see, for instance, F. T. Bizzarro et al. WO 00/58293) to generate a mixture of diastereoisomeric imides that are separable by any conventional method, e.g. column chromatography. Hydrolysis of the pure imides affords the stereopure (R)- and (S)-carboxylic acids that can then be condensed with heteroaryl amines III.

Alternatively stereopure (R)- and (S)-carboxylic acids II may be synthesized by enantioselective hydrogenation of the compound IV as described in WO2006/016178:

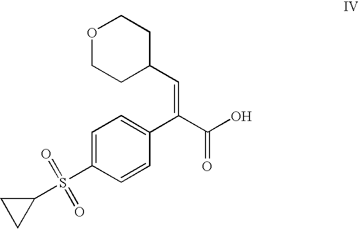

The hydrogenation of the compound is preferably conducted in the presence of a rhodium or ruthenium catalyst. The catalyst is preferably an anionic, neutral or cationic rhodium catalyst, more preferably a cationic rhodium catalyst. The catalyst is preferably generated in situ, for example from [Rh(nbd)₂]BF₄, [Rh(nbd)Cl]₂, or [Rul₂(α-cymeme)]₂ and a suitable ligand (nbd=norbornadiene).

Suitable ligands include diphosphine and phosphine ligands, preferably atropisomeric diphosphines, which may have additionally a chiral carbon atom (see M. Scalone Tetrahedron Asymmetry, 1997, 8, 3617; T. Uemura, J. Org. Chem., 1996, 61, 5510; and X. Zhang Synlett, 1994, 501), chiral diphosphine ligands such as for example Josiphos (EP-A-0612758), Walphos (F. Spindler, Adv. Synth. Catal., 2003, 345, 1; EP-A-1 1236994; and U.S. Pat. No. 6,777,567), Phospholane (CH0813/03), Mandyphos (EP-A-0965574; DE-A-1 19921924; and DE-A-1 19956374), Taniaphos (DE-A-1 19952348) and other ferrocene ligands such as for example Jafaphos (EP-A1-803510).

Particularly preferred are ferrocene ligands, for example Mandyphos ligands as described in EP-A-965574. Particular Mandyphos ligands which may be mentioned include (R)—(S)-MOD-Mandyphos and xyl-Mandyphos, especially (R)—(S)-MOD-Mandyphos (structure shown below):

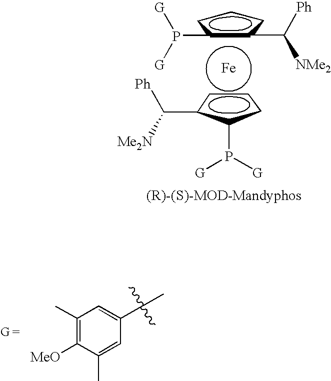

A particularly preferred catalyst/ligand combination is [Rh(nbd)₂]BF₄/(R)—(S)-MOD-Mandyphos.

Further details for the preparation of the compounds of Formula (I) are found in the examples.

During the synthesis of the compounds of Formula (I), labile functional groups in the intermediate compounds, e.g. hydroxy, oxo, carboxy and amino groups, may be protected. The protecting groups may be removed at any stage in the synthesis of the compounds of Formula (I) or may be present on the final compound of Formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

All publications, including, but not limited to, patents and patent application cited in this specification, are herein incorporated by reference as if each individual publication were specifically and individually indicated to be incorporated by reference herein as fully set forth.

### EXAMPLES

Abbreviations and acronyms: Ac: Acetyl; tBME: tert-Butylmethylether; ATP: Adenosine 5′-triphosphate; DCM: Dichloromethane; DMF: Dimethylformamide; Et: Ethyl; GK: Glucokinase; Glc: Glucose; G6P: Glucose-6-phosphate; G6PDH: Glucose-6-phosphate dehydrogenase; GST-GK: Glutathione S-transferase-Glucokinase fusion protein; NADP(H): β-Nicotinamide adenine dinucleotide phosphate (reduced); rt: Room temperature; THF: Tetrahydrofuran.

### Preparation 1

### Ethyl (4-cyclopropylsulfanylphenyl)oxoacetate

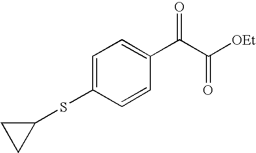

AlCl₃ (104.6 g, 0.79 mol) was suspended in CH₂Cl₂ (1.15 L) and cooled in an ice/salt bath to 0° C. with stirring. Ethyl chlorooxoacetate (84.8 g, 0.62 mol) was then added over a period of 10 min, during which time the temperature was maintained between 0 and 2° C. The mixture was then stirred for a further 30 min at 0° C., before the addition of cyclopropylphenylsulfide (85.0 g, 0.57 mol) over a period of 45 min, during which time the temperature remained between 0 and 8° C. The resulting mixture was allowed to warm to rt and stirred for a further 2 h. After this time ice/water (275 mL) was added, with ice bath cooling maintaining the temperature at 20° C. The organic layer was separated and washed with water (2×250 mL), saturated NaHCO₃ solution (2×250 mL) and again with water (1×250 mL). The organic fraction was dried (MgSO₄), filtered and the solvent removed to provide the title compound (134 g, 94% yield). NMR was consistent with the above structure.

### Preparation 2

### Ethyl (4-cyclopropylsulfonylphenyl)oxoacetate

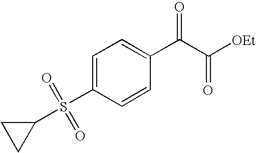

To a stirred solution of Preparation 1 (49.4 g, 0.2 mol) in CH₂Cl₂ (180 mL) was added a solution of m-chloroperoxybenzoic acid (92.0 g, 0.40 mol, calc as 75% strength) in CH₂Cl₂ (650 mL) over 45 min with the temperature maintained at 15-25° C. TLC(CH₂Cl₂:ethyl acetate 1:10) showed that starting material still remained. Further m-chloroperoxybenzoic acid (3.4 g) in CH₂Cl₂ was added and the reaction stirred for 30 min. A second TLC still showed the presence of some starting material, and additional m-chloroperoxybenzoic acid (3.4 g) was added and the reaction stirred for a further 2 h. TLC showed a small amount of starting material so a final quantity of m-chloroperoxybenzoic acid (1.0 g) was added and the reaction continued for 1 h. Sodium carbonate solution (2M, 500 ml) was then added and the aqueous layer was separated, the pH raised to 9-10 and reextracted with CH₂Cl₂. The organic extracts were combined, washed with water (2×400 ml), dried (MgSO₄), filtered and the solvent removed under vacuum (54.1 g, 96% yield). NMR was consistent with the above structure.

### Preparation 3

### (Tetrahydropyran-4-yl)methanol

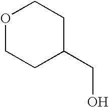

To a suspension of LiAlH₄ (56 g, 1.47 mol) in diethyl ether (2 L) under argon was added methyl tetrahydro-2H-pyran-4-carboxylate (270 g, 1.88 mol) in diethyl ether (ca. 200 mL) under reflux over a period of 1.75 h. After addition was complete reflux was continued for a further 1 h. TLC (diethyl ether) indicated a small amount of ester remained, so further LiAlH₄ (10 g, 0.26 mol) was added and reflux continued for 1 h. Water (66 mL) was added, then 15% NaOH solution (66 mL), followed by further water (198 mL). The solid was filtered and dried to give the crude product, which was redissolved in DCM (800 ml), dried (MgSO₄), filtered and the solvent removed to afford the title compound (207 g, 94% yield). NMR was consistent with the above structure.

### Preparation 4

### Methanesulfonic Acid (tetrahydropyran-4-yl)methyl ester

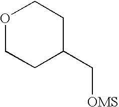

To a mixture of Preparation 3 (216.5 g, 1.87 mol) and triethylamine (299 mL) in DCM (1.3 L) at <10° C. was added under argon a solution of methanesulfonyl chloride (236 g, 160 mL) in DCM (200 mL) over 2 h 50 min, maintaining the temperature at 5-10° C. throughout. Subsequent washing with water (1 L), 1M HCl (500 mL), 5% NaHCO₃ (300 mL), water (300 mL), drying (MgSO₄) and then removal of the solvent afforded the title compound (328 g, 90% yield). NMR was consistent with the above structure.

### Preparation 5

### 4-Iodomethyltetrahydropyran

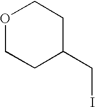

A mixture of Preparation 4 (328 g, 1.69 mol) and sodium iodide (507 g, 3.4 mol) in acetone (3.3 L) was refluxed for 4 h. TLC (diethyl ether) showed significant mesylate remaining so further sodium iodide (127 g, 0.65 mol) was added and reflux continued for 16 h. The mixture was cooled and filtered. The resulting cake was washed with acetone, dried, and then partitioned between diethyl ether (800 mL) and water (800 mL). The aqueous phase was re-extracted with diethyl ether (200 mL), the ether extracts combined and washed with 10% sodium thiosulphate solution (300 mL) which decolourised the extract. Final washing with water (300 mL), drying (MgSO₄) and then removal of the solvent provided the title compound (365 g, 92% yield). NMR was consistent with the above structure.

### Preparation 6

### Triphenyl(tetrahydropyran-4-ylmethyl)phosphonium iodide

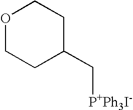

A mixture of Preparation 5 (350 g, 1.55M) and triphenylphosphine (406 g, 1.55M) in acetonitrile (1.6 L) was heated under reflux. After 27 h the mixture was cooled and filtered, washed with diethyl ether and dried in air to provide a white solid (504 g). Filtrate and washings were returned to reflux and concentrated to 750 mL, reflux was maintained for 16 h before cooling and dilution with diethyl ether (ca 1.2 L). A precipitate formed which was stirred for 30 min before being filtered, washed with diethyl ether (2×300 mL) and dried in air to yield a further crop (10 g). Overall yield of the title compound (604 g, 80%). R_{T}=2.7 min; m/z (ES⁺)=361.2.

### Preparation 7

### (E)-2-(4-Cyclopropanesulfonylphenyl)-3-(tetrahydropyran-4-yl)acrylic Acid

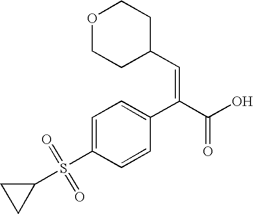

To a suspension of Preparation 6 (2.49 kg, 5.10 mol) in dry THF (5 L) maintained between −5 and 0° C. was added a solution of lithium hexamethyldisilazide (1.05M, 4.39 kg, 5.18 mol) over 30 min. The resulting mixture was then warmed to 15° C. and stirred for 2 h before recooling to between 0 and 5° C. A solution of Preparation 2 (1.25 kg, 4.43 mol) in THF (2.5 L) was then added over 1 h, during which time the temperature was maintained between 0 and 5° C., before a period of 16 h at between 20 and 25° C. Subsequently, brine (17% w/w, 3.8 L) was added and the phases separated with the aid of additional brine (1.3 L). The aqueous phase was reextracted with methyl t-butyl ether (2×2.5 L) and the combined organic extracts washed with brine (2×3.8 L). The solvents were removed under vacuum at between 30 and 40° C. The residue was dissolved in methanol (15 L) and aqueous sodium hydroxide (2M, 4.34 L) added before heating at 65-67° C. for 4 h. The mixture was cooled and the solvents removed under vacuum at between 35 and 40° C. until water started to distil. The residue was diluted with water (15 L). The solid phosphine oxide was filtered off, washed with water (2.5 L) and the filtrate separated. The aqueous phase was washed with methyl t-butyl ether (5 L and 3.5 L), before acidification with hydrochloric acid solution (5M, 1.9 L) in the presence of methyl t-butyl ether (10 L). The organic phase was separated and the aqueous phase reextracted with methyl t-butyl ether (5 L). The combined organic extracts were washed with saturated brine (2×1 L) and the solvent removed under vacuum. Methanol (2 L) was added and then removed under vacuum, this step was then repeated. The residue was brought to a total weight of 4.0 kg by addition of methanol and stirred at ambient temperature to crystallise the product. Filtration of the solid and washing with chilled (ca 0° C.) methanol (500 mL) gave, after vacuum drying at 40° C., the title compound (654 g, 41% yield after correction for residual solvent). NMR was consistent with the above structure.

### Preparation 8

### (2R)-2-(4-Cyclopropanesulfonylphenyl)-3-(tetrahydropyran-4-yl)propionic Acid

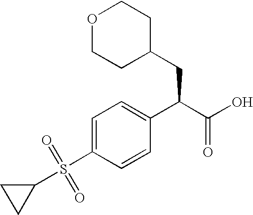

(E)-2-(4-Cyclopropanesulfonylphenyl)-3-(tetrahydropyran-4-yl)acrylic acid (Preparation 7, 110 g, 0.327 mol) was dissolved in MeOH/Toluene 5:1 (1.4 L). In a 40 mL Schlenk flask was placed [Rh(nbd)₂](BF₄) (30.5 mg, 0.08 mmol) and All-MOD-Mandyphos (90.4 mg, 0.08 mmol), dissolved in MeOH (10 mL) and stirred for 1 h at rt. This catalyst solution was then added to the (E)-2-(4-cyclopropanesulfonylphenyl)-3-(tetrahydropyran-4-yl)acrylic acid solution and transferred to a 2.5 L autoclave. The autoclave was pressurized to 50 bar and heated to 30° C. After 18 h the pressure was released and the solution transferred to a 3 L flask. Active charcoal (3 g) was added to the reaction mixture, stirred for 1 h and the charcoal removed by filtration. The solution was further filtered over Hyflo and a Zeta-Bond filter. The solution thus obtained was concentrated under partial pressure and the solid obtained further dried under high vacuum to give a solid (105 g). The solid was placed in a 1.5 L flask equipped with a mechanical stirrer, a thermometer and a dropping funnel. Isobutylacetate (540 mL) was added at rt and the suspension heated at 110° C. until a clear solution was observed. Heptane (60 mL) was added slowly at 110° C., the oil bath was then removed and the solution allowed to cool slowly. The reaction was stirred for a further 16 h, the title compound filtered off and dried under high vacuum (77.2 g, 70% yield, 99% ee). ¹H NMR (CDCl₃, 300.13 MHz) δ: 7.85 (2H, Aryl H. d, JHH=6.6 Hz), 7.50 (2H, Aryl H. d, JHH=6.6 Hz), 3.95 (br d, 2H), 3.80 (t, 1H, CHCH₂, JHH=7.8 Hz), 3.35 (m, 2H), 2.45 (m, 1H), 2.10 (m, 1H), 1.75 (m, 1H), 1.60 (m, 2H), 1.50-1.20 (m, 5H), 1.05 (m, 2H).

### Preparation 9

### 2-(4-Cyclopropanesulfonylphenyl)-3-(tetrahydropyran-4-yl)propionic Acid

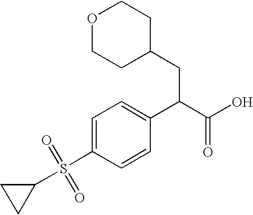

A stirred suspension of AlCl₃ (12.90 g, 96.8 mmol) in anhydrous CH₂Cl₂ (135 mL) was treated portionwise at 0° C. with ethyl chlorooxoacetate (8.5 mL, 76.0 mmol). Cyclopropyl phenyl sulfide (10.0 mL, 70.0 mmol) was added to the mixture dropwise over 1 h while maintaining the reaction temperature below 10° C. The mixture was allowed to warm to 20° C., before being stirred for an additional 70 min. Ice cold H₂O (35 mL) was added on cooling to 0° C., then the mixture was stirred further for 10 min. The CH₂Cl₂ layer was separated, then the aqueous layer was extracted with more CH₂Cl₂ (2×50 mL). The combined organic layers were dried (MgSO₄), filtered and concentrated to give ethyl (4-cyclopropylsulfanylphenyl)-oxoacetate: RTB=1.74 min. LHMDS (3.7 mL of a 1.0M solution in THF, 3.7 mmol) was added to a stirred suspension of triphenyl(tetrahydropyran-4-ylmethyl)phosphonium iodide (Preparation 6, 1.82 g, 3.7 mmol) in anhydrous THF (5.6 mL) at 0° C. After 1 h, a solution of ethyl (4-cyclopropylsulfanylphenyl)oxoacetate (0.78 g, 3.1 mmol) in anhydrous THF (4 mL) was added over 5 min. The mixture was stirred at 0° C. for 1 h, before being allowed to warm to 20° C. over 16 h. H₂O (7 mL) was added on cooling down to 0° C. 1M HCl was added to adjust the pH to 6, then the mixture was stirred for 1 h at 20° C. The THF was removed in vacuo, then Et₂O (35 mL) was added. The mixture was stirred for 30 min and filtered, washing with Et₂O. The aqueous layer was separated and extracted with Et₂O (3×10 mL). The combined organic extracts were washed with brine (20 mL), dried, filtered, and concentrated. Flash chromatography (IH—CH₂Cl₂, 2:1 to 1:1, followed by THF—CH₂Cl₂, 1:99) yielded ethyl 2-(4-cyclopropylsulfanylphenyl)-3-(tetrahydropyran-4-yl)acrylate: m/z (ES⁺)=333.2 [M+H]⁺. A stirred solution of this thioether (609 mg, 1.83 mmol) in CH₂Cl₂ (35 mL) was treated with a solution of mCPBA (992 mg of 65% pure, 3.74 mmol) in CH₂Cl₂ (15 mL). After 16 h, saturated aqueous NaHCO₃ (25 mL) was added, then stirring was continued for 5 min. The layers were separated, then the aqueous phase was extracted with CH₂Cl₂ (20 mL). The combined organic layers were washed with saturated aqueous NaHCO₃ (25 mL), H₂O (25 mL), and brine (25 mL), before being dried (MgSO₄). Filtration and solvent evaporation gave ethyl 2-(4-cyclopropanesulfonylphenyl)-3-(tetrahydropyran-4-yl)acrylate: m/z (ES⁺)=382.2 [M+NH₄]⁺. A solution of this compound (667 mg, 1.83 mmol) in EtOAc (60 mL) was treated with Pd (10% on C, 424 mg, 0.39 mmol). The reaction mixture was stirred under a H₂ atmosphere for 3 d, before being filtered through Celite. The Celite was washed with EtOAc (100 mL), then the combined filtrates were concentrated to give ethyl 2-(4-cyclopropane-sulfonylphenyl)-3-(tetrahydropyran-4-yl)propionate: RF (CH₂Cl₂-THF, 30:1)=0.56. A solution of this ester (664 mg, 1.81 mmol) in THF—H₂O (3:1, 20 mL) was stirred with LiOH.H₂O (168 mg, 4.00 mmol) for 23 h. The THF was evaporated off under reduced pressure, then the remainder was diluted with H₂O (100 mL). The mixture was washed with Et₂O (2×20 mL), before being acidified with 2M HCl (5 mL) to pH1. The remainder was extracted with EtOAc (3×20 mL). The combined organic extracts were washed with brine (20 mL), dried (MgSO₄), filtered, and evaporated to give the title compound: m/z (ES⁺)=694.4 [2M+NH₄]⁺.

### Examples

(2R)-2-(4-Cyclopropanesulfonylphenyl)-3-(tetrahydropyran-4-yl)propionic acid (Preparation 8) was coupled with amines selected from 2-amino-5-methylpyrazine, 2-amino-5-methylpyridine, 2-amino-5-chloropyridine, 2-aminopyridine, 3-amino-5-methylisoxazole, 3-aminoisoxazole, 2-amino-5-methylthiazole, 3-amino-6-methylpyridazine, 1-methyl-3-aminopyrazole and 4-aminopyrimidine using the following procedure to provide Examples 1-10.

CH₂Cl₂ (60 mL) and DMF (0.08 mL, 1.064 mmol, 1.2 eq) were cooled to −10° C. and oxalylchloride slowly added (0.09 mL, 0.465 mol, 1.2 eq). After stirring for 15 min the reaction mixture was cooled to −30° C. and (2R)-2-(4-cyclopropanesulfonylphenyl)-3-(tetrahydropyran-4-yl)propionic acid (Preparation 8, 0.300 g, 0.886 mmol, 1.0 eq) was added. The reaction was stirred at −30° C. for 45 min then pyridine (1.395 mol, 0.31 mL in 1 mL CH₂Cl₂, 4.5 eq) and the amine (4.43 mmol, 5.0 eq) were slowly added in parallel at −40° C. The reaction mixture was stirred for 15 min then the ice bath removed. The reaction mixture was stirred for 2 h until it reached rt. The solvent was removed under partial vacuum and the crude mixture dissolved in EtOAc (10 mL) and aqueous HCl (1.5 mL). The layers were separated and the aqueous phase extracted with EtOAc (5 mL). The organic fractions were combined and washed with H₂O (10 mL), saturated aqueous NaHCO₃ (2×10 mL), water (5 mL) and brine (5 mL) and dried (Mg₂SO₄). Purification was by flash chromatography (EtOAc:heptane, 2:1) and/or recrystallisation.

| | | | ¹H-NMR δ_{H} (CDCl₃) |
|---|---|---|---|
| Eg | Structure | Name | m/z (ES⁺) |
| 1 | 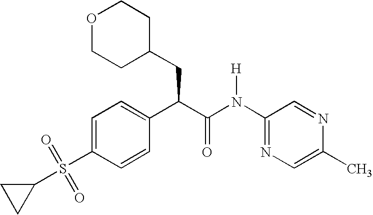 | (R)-2-(4-Cyclopropanesulfonylphenyl)-N-(5-methylpyrazin-2-yl)-3-(tetrahydropyran-4-yl)propionamide | 0.90-1.00 (m, 2H), 1.20-1.45 (m, 5H), 1.50-1.63(m, 2H), 1.70-1.80 (m, 1H), 2.10-2.20 (m, 1H),2.36-2.46 (m, 4H), 3.20-3.30 (m, 2H), 3.70-3.75(m, 1H), 3.80-3.90 (m, 2H), 7.49 (d, 2H), 7.82 (d,2H), 7.87 (s, 1H), 8.01 (s, 1H), 9.33 (s, 1H)430 [M + H]⁺ |
| 2 | 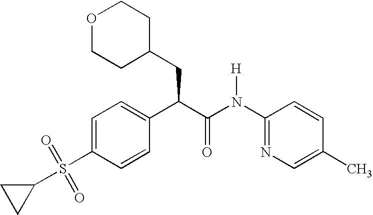 | (R)-2-(4-Cyclopropanesulfonylphenyl)-N-(5-methylpyridin-2-yl)-3-(tetrahydropyran-4-yl)propionamide | 0.90-1.00 (m, 2H), 1.20-1.42 (m, 5H), 1.50-1.60(m, 2H), 1.65-1.75 (m, 1H), 2.10-2.21 (m, 4H),2.35-2.45 (m, 1H), 3.19-3.28 (m, 2H), 3.60-3.70(m, 1H), 3.80-3.90 (m, 2H), 7.43-7.49 (m, 3H),7.80 (d, 2H), 7.97-8.03 (m, 3H)429 [M + H]⁺ |
| 3 | 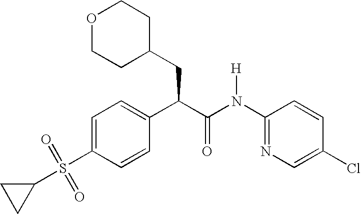 | (R)-2-(4-Cyclopropanesulfonylphenyl)-N-(5-chloropyridin-2-yl)-3-(tetrahydropyran-4-yl)propionamide | 0.92-1.02 (m, 2H), 1.20-1.40 (m, 5H), 1.50-1.60(m, 2H), 1.70-1.79 (m, 1H), 2.06-2.20 (m, 1H),2.35-2.45 (m, 1H), 3.18-3.29 (m, 2H), 3.61-3.70(m, 1H), 3.80-3.90 (m, 2H), 7.47 (d, 2H), 7.60 (dd,1H), 7.82 (d, 2H), 7.92 (br s, 1H), 8.04-8.15 (m,2H)449 [M + H]⁺ |
| 4 | 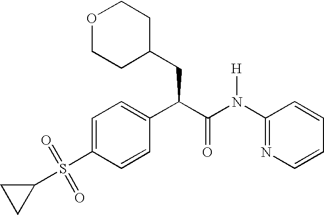 | (R)-2-(4-Cyclopropanesulfonylphenyl)-N-(pyridin-2-yl)-3-(tetrahydropyran-4-yl)propionamide | 0.90-1.00 (m, 2H), 1.18-1.42 (m, 5H), 1.50-1.60(m, 2H), 1.65-1.75 (m, 1H), 2.09-2.20 (m, 1H),2.35-2.45 (m, 1H), 3.20-3.30 (m, 2H), 3.65-3.75(m, 1H), 3.80-3.90 (m, 2H), 7.00 (m, 1H), 7.47 (d,2H), 7.67 (m, 1H), 7.80 (d, 2H), 8.12-8.18 (m, 2H),8.33 (br, 1H)415 [M + H]⁺ |
| 5 | 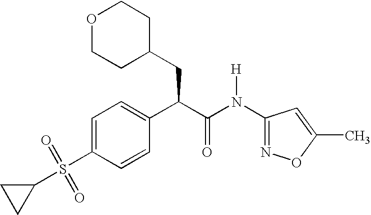 | (R)-2-(4-Cyclopropanesulfonylphenyl)-N-(5-methylisoxazol-3-yl)-3-(tetrahydropyran-4-yl)propionamide | 0.90-1.00 (m, 2H), 1.20-1.42 (m, 5H), 1.50-1.62(m, 2H), 1.70-1.80 (m, 1H), 2.09-2.20 (m, 1H),2.30-2.40 (m, 4H), 3.19-3.26 (m, 2H), 3.80-3.90(m, 3H), 6.73 (s, 1H), 7.52 (d, 2H), 7.77 (d, 2H),10.25 (s, 1H)419 [M + H]⁺ |
| 6 | 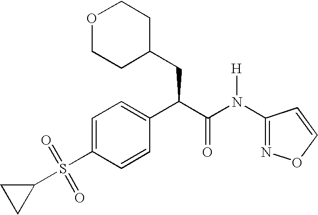 | (R)-2-(4-Cyclopropanesulfonylphenyl)-N-(isoxazol-3-yl)-3-(tetrahydropyran-4-yl)propionamide | 0.90-1.00 (m, 2H), 1.18-1.40 (m, 5H), 1.50-1.65(m, 2H), 1.70-1.80 (m, 1H), 2.08-2.20 (m, 1H),2.30-2.40 (m, 1H), 3.17-3.25 (m, 2H), 3.80-3.90(m, 3H), 7.10 (d, 1H), 7.52 (d, 2H), 7.78 (d, 2H),8.30 (d, 1H), 10.16 (br, 1H)405 [M + H]⁺ |
| 7 | 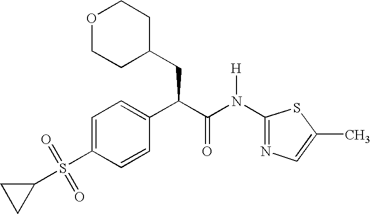 | (R)-2-(4-Cyclopropanesulfonylphenyl)-N-(5-methylthiazol-2-yl)-3-(tetrahydropyran-4-yl)propionamide | 0.90-1.00 (m, 2H), 1.18-1.40 (m, 5H), 1.50-1.60(br, 2H), 1.70-1.80 (m, 1H), 2.10-2.22 (m, 1H),2.30-2.40 (m, 4H), 3.18-3.27 (m, 2H), 3.79-3.88(m, 3H), 7.05 (s, 1H), 7.40 (d, 2H), 7.74 (d, 2H)435 [M + H]⁺ |
| 8 | 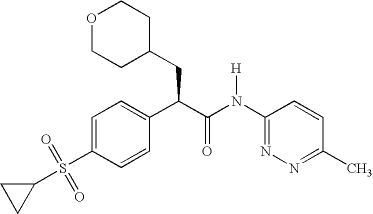 | (R)-2-(4-Cyclopropanesulfonylphenyl)-N-(6-methylpyridazin-3-yl)-3-(tetrahydropyran-4-yl)propionamide | 0.89-0.99 (m, 2H), 1.20-1.45 (m, 5H), 1.60-1.85(m, 3H), 2.10-2.20 (m, 1H), 2.30-2.40 (m, 1H),2.62 (s, 3H), 3.17-3.26 (m, 2H), 3.77-3.86 (m, 2H),4.72-4.77 (m, 1H), 7.40 (d, 1H), 7.63 (d, 2H), 7.72(d, 2H), 8.54 (d, 1H), 11.59 (br s, 1H)430 [M + H]⁺ |
| 9 | 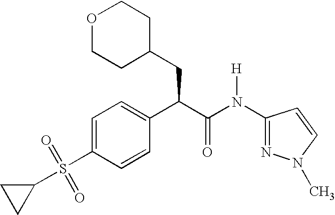 | (R)-2-(4-Cyclopropanesulfonylphenyl)-N-(1-methylpyrazol-3-yl)-3-(tetrahydropyran-4-yl)propionamide | 0.90-1.00 (m, 2H), 1.20-1.45 (m, 5H), 1.50-1.60(br m, 2H), 1.70-1.80 (s, 1H), 2.10-2.20 (m, 1H),2.35-2.45 (m, 1H), 3.18-3.27 (m, 2H), 3.65-3.72(m, 4H), 3.80-3.90 (m, 2H), 6.61 (d, 1H), 7.20 (s,1H), 418 [M + H]⁺ 7.51 (d, 2H), 7.80 (d, 2H), 8.32(d, 1H) |
| 10 | 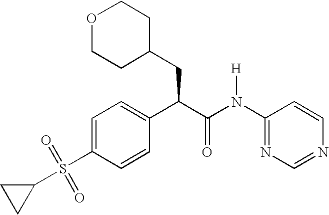 | (R)-2-(4-Cyclopropanesulfonylphenyl)-N-(pyrimidin-2-yl)-3-(tetrahydropyran-4-yl)propionamide | 0.90-1.00 (m, 2H), 1.18-1.45 (m, 5H), 1.50-1.61(m, 2H), 1.70-1.80 (m, 1H), 2.10-2.19 (m, 1H),2.35-2.45 (m, 1H), 3.18-3.27 (m, 2H), 3.80-3.95(m, 3H), 7.48 (d, 2H), 7.82 (d, 2H), 8.20 (d, 1H),8.57 (d, 1H), 8.87 (s, 1H), 9.02 (br s (1H)416 [M + H]⁺ |

### Assays

### In Vitro GK Activity

Using a protocol similar to that described in WO2000/58293, GK activity was measured by coupling the production of G6P by GST-GK to the generation of NADH with G6PDH as the coupling enzyme.

The assay was performed at room temperature (23° C.) in clear flat bottom 96-well plates in a total volume of 100 μl consisting of 25 mM Hepes (pH 7.4), 25 mM KCl, 5 mM D-glucose, 1 mM ATP, 1 mM NADP, 2 mM MgCl₂, 1 mM dithiothreitol, 0.2 μg purified GST-GK derived from human liver GK and a range of activator concentrations in a final concentration of 5% DMSO. The incubation time was 15 min at which time the reaction has been shown to be linear. The generation of NADH, as an indirect determination of GK activity, was measured at OD₃₄₀ in a SpectraMAX 190 microplate spectrophotometer (Molecular Devices Corp).

Typically compounds were tested over a range of 10 dilutions from 100 μM to 0.004 μM in a final DMSO concentration of 5%. The degree of activation was calculated as a ratio over a control reaction with 5% DMSO only. Values quoted represent the concentration of compound required to produce a 2-fold activation of GK derived from a dose response curve constructed using a 4-parameter logistic model. Additionally, maximum fold activation and an EC₅₀ (concentration required to produce half the maximum fold activation) was calculated from the same dose response curve.

Representative examples of the compounds of Formula (I) had EC₅₀s of <500 nM.

### In Vivo GK Activity (I)

Following a 4.5 h fasting period, C57BL/6 mice were dosed orally via gavage with GK activator at 10 mg/kg body weight followed by a glucose load of 2 g/kg. Blood Glc determinations were made 3 times during the 2.5 h post-dose study period.

Mice (n=9) were weighed and fasted for 4.5 h before oral treatment. GK activators were dissolved in Gelucire 44/14-water (1:9 v/v) at a concentration of 1 mg/mL. Mice were dosed orally with 10 mL formulation per kg of body weight to equal a 10 mg/kg dose. Fifteen min prior to dosing, a pre-dose blood Glc reading was acquired by snipping off a small portion of the animals' tails (<1 mm) and collecting 20 μL blood for analysis. After GK activator treatment, further blood Glc readings were taken at 0.5, 1.0, and 2.5 h post-dose from the same tail wound. Results were interpreted by comparing the mean blood Glc values of the vehicle treated mice with the GK activator treated mice over the study duration. Representative examples of the compounds of Formula (I) exhibited a statistically significant decrease in blood Glc compared to vehicle for 2 consecutive assay time points following compound administration.

### In Vivo GK Activity (II)

The antihyperglycaemic effects of examples of the GK activators of the invention were evaluated in oral glucose tolerance tests in 7-8 week old male C57B1/6 ob/ob mice. Briefly, mice (n=6) were weighed and their basal blood glucose levels determined from 20 μL of blood withdrawn from a tail cut (T-27 h). After 22 h (T-5 h), food was removed and the mice were placed in fresh cages with access to water ad libitum. The blood glucose levels were determined at T-0.75 h from 20 μL of blood withdrawn from the tail wound. The GK activators were dissolved in a Gelucire 44/14-water (1:9 v/v) mixture at a concentration of 1 mg/mL, then, at T-0.5 h, the mice were dosed orally with 10 mL formulation per kg of body weight to equal a 10 mg/kg dose. At T=0 h, the mice were bled (20 μL) for analysis of blood glucose levels, then immediately dosed orally with glucose (2 g/kg). Further blood samples (20 μL) were taken from each animal at T=+0.5, +1.0, +1.5, +2.0, +3.0, and +4.0 h for the analysis of glucose levels. Representative examples of the compounds of Formula (I) typically reduced the area under the glucose curve by at least 20% in the 2 h following administration of glucose.

1. A compound of Formula (I): 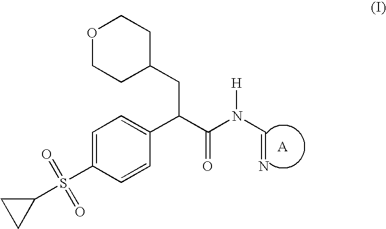
wherein A is a nitrogen containing heteroaryl ring selected from 5-methylpyrazin-2-yl, 5-methylpyrid-2-yl, 5-chloropyrid-2-yl, pyrid-2-yl, 5-methylisoxazol-3-yl, isoxazol-3-yl, 5-methylthiazol-2-yl, 6-methylpyridazin-3-yl, 1-methylpyrazol-3-yl and pyrimidin-4-yl;
or a pharmaceutically acceptable salt thereof.

2. A compound according to claim 1, or a pharmaceutically acceptable salt thereof, wherein the carbon atom linking the phenyl ring and the tetrahydropyran containing sidechain to the amide carbonyl carbon is in the (R)-configuration.

3. A compound according to claim 1, or a pharmaceutically acceptable salt thereof, wherein A represents 5-methylpyrazin-2-yl.

4. A compound according to claim 1, or a pharmaceutically acceptable salt thereof, wherein A represents 5-methylpyrid-2-yl.

5. A compound according to claim 1, or a pharmaceutically acceptable salt thereof, wherein A represents 5-chloropyrid-2-yl.

6. A compound according to claim 1, or a pharmaceutically acceptable salt thereof, wherein A represents pyrid-2-yl.

7. A compound according to claim 1 or a pharmaceutically acceptable salt thereof, wherein A represents 5-methylisoxazol-3-yl.

8. A compound according to claim 1, or a pharmaceutically acceptable salt thereof, wherein A represents isoxazol-3-yl.

9. A compound according to claim 1, or a pharmaceutically acceptable salt thereof, wherein A represents 5-methylthiazol-2-yl.

10. A compound according to claim 1, or a pharmaceutically acceptable salt thereof, wherein A represents 6-methylpyridazin-3-yl.

11. A compound according to claim 1, or a pharmaceutically acceptable salt thereof, wherein A represents 1-methylpyrazol-3-yl.

12. A compound according to claim 1, or a pharmaceutically acceptable salt thereof, wherein A represents pyrimidin-4-yl.

13. A pharmaceutical composition comprising a compound according to claim 1, or a pharmaceutically acceptable salt thereof, and a pharmaceutically acceptable carrier.

14. A method of therapeutic treatment of a condition where activation of GK is desirable comprising a step of administering an effective amount of a compound according to claim 1, or a pharmaceutically acceptable salt thereof.

15. A method of according to claim 14 wherein the condition where activation of GK is desirable is hyperglycemia or diabetes.

16. A method according to claim 15 wherein the compound according to claim 1, or a pharmaceutically acceptable salt thereof, is administered in combination with one or more other anti-hyperglycemic agents or anti-diabetic agents.

17. A method of prevention of diabetes in a human demonstrating pre-diabetic hyperglycemia or impaired glucose tolerance comprising a step of administering an effective prophylactic amount of a compound according to claim 1, or a pharmaceutically acceptable salt thereof.

18. A process for the preparation of a compound of Formula (I): 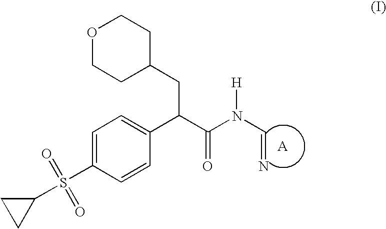
or a pharmaceutically acceptable salt thereof, said process comprising the condensation of a compound of Formula (II) or an activated derivative thereof: 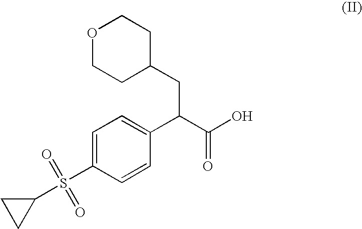
with a compound of Formula (III): 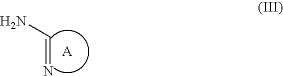
or a salt thereof, wherein A is as defined in claim 1.
